(19) United States
(12) Patent Application Publication
(10) Pub. No.: US 2007/0059775 A1
(43) Pub. Date: Mar. 15, 2007

(54) Synthesis and conjugation of iron oxide nanoparticles to antibodies for targeting specific cells using fluorescence and MR imaging techniques

(75) Inventors: Kristi Hultman, New York, NY (US); Amanda Willis, New York, NY (US); Stephen O'Brien, New York, NY (US); Truman R. Brown, New York, NY (US); Paul Harris, New York, NY (US); Nicholas J. Turro, Tenafly, NJ (US); Adrienne L. Grzenda, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/394,040

(22) Filed: Mar. 29, 2006

Related U.S. Application Data
(60) Provisional application No. 60/665,963, filed on Mar. 29, 2005.

(51) Int. Cl. G01N 33/567; G01N 33/551; G01N 33/53

(52) U.S. Cl. 435/7.2; 436/524; 977/902

(57) ABSTRACT

The invention provides for methods for producing water-soluble iron oxide nanoparticles comprising encapsulating the nanoparticles in phospholipids micelles. Also provided are methods for conjugating the inventive nanoparticles via functionalized phospholipids to a target molecule, such as an antibody. The invention further provides methods for using the nanoparticle-antibody conjugate of the invention as a contrast agent to image specific cells or proteins in a subject using fluorescent and magnetic imaging techniques.

This application claims priority to U.S. Application No. 60/665,963, filed on Mar. 29, 2005, which is hereby incorporated by reference in its entirety.

The invention disclosed herein was made with U.S. Government support under NIH and NSF Grant Nos. 5 POI CA41078-14, DMR-0213574, and CHE-0117752. Accordingly, the U.S. Government may have certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

Noninvasive detection and imaging of tumors and specific organs can currently be carried out with contrast agents and imaging technology, such as fluorescence and magnetic resonance imaging (MRI). These imaging techniques are important in diagnosing and treating various diseases. However, the ability to detect specific cells or molecules inside cells, such as proteins and nucleic acids, is a new and developing technology. Dextran-coated nanoparticles have been conjugated to biological molecules and used as a contrast agent to detect or image cells in vivo. Problems encountered with dextran-coated nanoparticles include the large size of the dextran coating, the high level of purification required, and the potential f;r immune reactions triggered by the dextran coating. Therefore, it is important to develop contrast agents that are easier to prepare and are more amenable to use in humans, including diagnosis, treatment and other biomedical and therapeutic applications.

### SUMMARY OF THE INVENTION

The invention provides a method for producing an iron oxide nanoparticle, the method comprising injecting iron pentacarbonyl into a reaction mixture, wherein the reaction mixture comprises oleic acid and trioctylamine (TOA), and wherein the reaction mixture is at a temperature of from about 180° C. to about 220° C. In one embodiment of the invention, the reaction mixture is at a temperature of from about 190° C. to about 210°C. In another embodiment, the reaction mixture is at a temperature of from about 195° C. to about 205° C. In a further embodiment, the reaction mixture is at a temperature of from about 198° C. to about 202° C. In a specific embodiment, the reaction mixture is at a temperature of about 200° C.

In an embodiment of the method of the invention, the reaction mixture consists of oleic acid and trioctylamine (TOA). In another embodiment, the reaction mixture consists essentially of oleic acid and trioctylamine (TOA).

An embodiment of the inventive method further encompasses encapsulating the iron oxide nanoparticle in a phospholipid micelle, thereby making the iron oxide nanoparticle water-soluble. In an additional embodiment, the nanoparticle is from about 2 to about 20 nanometers. In a specific embodiment, the nanoparticle is about 5 nanometers. In yet another embodiment, the nanoparticle comprises maghemite. In another embodiment of the method of the invention, the micelle comprises polyethylene glycol, methoxypolyethylene glycol 2000 (mPEG 2000), mPEG 2000 maleimide, 1,2-Diacyl-sn-Glycero-3-Phosphoethanolamine-N-[Methoxy(Polyethylene glycol)-350], 1,2-Diacyl-sn-Glycero-3-Phosphoethanolamine-N-[Methoxy(Polyethylene glycol)-750], 1,2-Diacyl-sn-Glycero-3-Phosphoethanolamine-N-[Methoxy(Polyethylene glycol)-2000], 1,2-Dipalmitoyl-sn-Glycero-3-Phosphocholine, cholesterol, or any combination thereof.

In yet another embodiment, the micelle comprises from about 0.1% to about 10% functionalized phospholipids. In a specific embodiment, the micelle comprises about 1% functionalized phospholipids. In another embodiment of the inventive method, the functionalized phospholipids comprise thiol-functionalized phospholipids, amine functionalized phospholipids, or any combination thereof. In another embodiment, the amine functionalized phospholipids comprise DSPE-PEG(2000)Carboxylic Acid, DSPE-PEG(2000)Maleimide, DSPE-PEG(2000)PDP, DSPE-PEG(2000)Amine, DSPE-PEG(2000)Biotin, or any combination thereof. In yet another embodiment, the thiol-functionalized phospholipids comprise phophatidylthioethanol (PTE). In a further embodiment, the micelle comprises about 99% mPEG750 and about 1% PTE phospholipids.

In another embodiment of the method, the micelle further comprises phospholipids labeled with a fluorescent marker. In one embodiment, the fluorescent marker comprises fluorescein. In a specific embodiment, phospholipids comprise 1,2-dioleoyl-sn-glycero-3-phosphoethanolamine-N-(Lissamine Rhodamine B Sulfonyl).

The invention also provides a method for conjugating a water-soluble iron oxide nanoparticle to a target molecule, the method comprising (a) reacting a target molecule with a crosslinking agent, thereby forming a target molecule-crosslinking agent complex; and (b) reacting a water-soluble iron oxide nanoparticle to the complex of step (a). In one embodiment, the method comprises concentrating the complex of step (a) before performing step (b). In another embodiment, the crosslinking agent comprises a heterobifunctional crosslinking agent. In yet another embodiment, the crosslinking agent comprises SMPT (4-succinimidyloxycarbonyl-α-methyl-α-(2-pyridylditio)toluene), sulfo-LC-SMPT (sulfosuccinimidyl-6-(α-methyl-α-(2-pyridylthio)toluamido) hexanoate, Traut's reagent (2-Iminothiolane•HCl), or any combination thereof.

The invention further encompasses a method for conjugating a water-soluble iron oxide nanoparticle to a target molecule in the absence of a crosslinking agent, wherein the nanoparticle is conjugated directly to the target molecule.

In certain embodiments of the methods of the invention, the target molecule comprises a therapeutic agent. In other embodiments, target molecule comprises a polypeptide, a nucleic acid, or a small molecule. In additional embodiments, the target molecule comprises a antibody. In a specific embodiment, the antibody comprises an anti-insulin antibody.

The invention provides a nanoparticle-target molecule conjugate prepared by one of the methods of the invention.

The invention also provides a method for detecting a cell of interest in a subject, the method comprising administering to the subject an effective amount of an iron oxide nanoparticle-antibody conjugate, wherein the antibody specifically binds to the cell. In one embodiment, the nanoparticle is detected by magnetic resonance imaging or fluorescence imaging.

The invention additionally provides a method for detecting a polypeptide in a subject, the method comprising administering to the subject an effective amount of an iron oxide nanoparticle-antibody conjugate, wherein the antibody specifically binds to the polypeptide. In one embodiment, the nanoparticle is detected by magnetic resonance imaging or fluorescence imaging.

### BRIEF DESCRIPTION OF THE DRAWINGS

FIGS. 1A-1B. Transmission electron microscopy (TEM) images of γ-Fe₂O₃ nanoparticles in chloroform magnified 150,000 times with diameters of 13 nm (FIG. 1A), and 8.7 nm (FIG. 1B). The nanoparticles are uniform in size and shape and self-organize into 1-dimensional lattice structures. The nanoparticles are uniform in size and shape and self-organize into 1-dimensional lattice on the copper grid. Both samples were made using the same procedure and amounts, however, notice how the nanoparticles in FIG. 1B are smaller than the nanoparticles in FIG. 1A.

FIG. 2. Four nanometer γ-Fe₂O₃ nanoparticles coated with 60% DPPC and 40% mPEG2000 in water. Notice the white phospholipid ring around the outside of each nanoparticles. Notice how the nanoparticles appear more separated from their surrounding particles and like that have a white ring around them, this is the layer of phospholipids coating the nanoparticles. The nanoparticles also appear less organized as they appeared in the chloroform solution (see FIG. 1). This is a result of the polar quality of tne water interacting with the nanoparticles.

FIGS. 3A-3B. γ-Fe₂O₃ nanoparticles coated with 20% PTE; note the aggregation (FIG. 3A). γ-Fe₂O₃ nanoparticles coated with 1% PTE, no noticeable aggregation between particles (FIG. 3B). In FIG. 3A, the nanoparticles are coated with 20% PTE and have aggregated into a ball on the grid. In solution, the nanoparticles precipitated to the bottom of the vial rather than being dispersed throughout the water. The nanoparticles coated with only 1% PTE, FIG. 3B, are dispersed uniformly throughout the solution.

FIG. 4. A T2 scan of a series of 8 dilutions of γ-Fe₂O₃ nanoparticles in gelatin for varying TE times, notice how the intensity of the spot decreased as the concentration of the particles increases and as the TE time increases. The intensity of the spot also decreases as the TE time increase, especially for concentrations above 10 MIONs/μm³. For the highest concentrations, the spots are almost impossible to detect for TE times above 200 ms.

FIG. 5. Semilog plot of intensity verses TE time for 4 nm MIONs. Note that the slope, equal to the negative of the R2 value, decreases as the concentration increases.

FIG. 6. Relaxation rate versus concentration of 4 nm MIONs for three separate CPMG scans. The plot appears linear for low concentrations and then increases exponentially above 10 MIONs/μm³. The scans were conducted by scanning the same sample over a period of five weeks, one week between scan 1 and scan 2, and a month between scan 2 and scan three. The relaxation rates for the various concentrations remained very consistent and do not appear to change significantly between scans or over time.

FIG. 7. MRI CPMG scans of 8 nm and 10 nm dilutions for various TE times, notice the decrease in intensity for concentrations above 10 MIONs/μm³ and for TE times above 100 ms.

FIG. 8. Intensity versus TE time plots for 8 and 10 nm dilution samples, notice that the intensity drops off rapidly for concentrations above 20 MIONs/μm³, and that the curves are almost identical despite different sizes.

FIG. 9. Relaxation times versus concentration for 4, 8, and 10 nm dilutions. All three curves exhibit a similar shape, despite the lower magnitude of the 4 nm curve.

FIG. 10. γ-Fe₂O₃ nanoparticles coated with 10 , 20, 30 or 40 μL Fluorescein-tagged phospholipids in addition to mPEG750 and PTE. No significant difference in the intensity of the fluorescence is observed.

FIG. 11. Pancreas tissue incubated with 100 μL of Fluorescein-tagged nanoparticles for 15, 30, 45 and 60 min. The lack of green spots or a green tinge indicates that there are no nanoparticles present after the rinsing procedure, and thus no unwanted interactions.

FIG. 12. Anti-insulin immunohistochemistry stain for beta cells in the pancreas using a Texas Red conjugated secondary antibody. The stain is very clean with little background.

FIGS. 13A-13F. Fluorescein-tagged nanoparticles conjugated to Texas Red tagged secondary antibodies (FIGS. 13A-13C). Texas Red-tagged secondary antibodies (FIGS. 13D-13F). FIGS. 13A and 13D have all channels shown; FIGS. 13B and 13E have the red channel removed; FIGS. 13C and 13F have the green channel removed.

FIGS. 14A-14B. IHC assays of human pancreas tissue stained using a guinea pig anti-insulin primary antibody and either a Texas Red conjugated anti-guinea pig secondary antibody (FIG. 14A) or a MION conjugated anti-guinea pig secondary antibody (FIG. 14B). The staining patterns of the islets in the tissue are identical, indicating that the conjugation is successful and does not interfere with the functioning of the antibody.

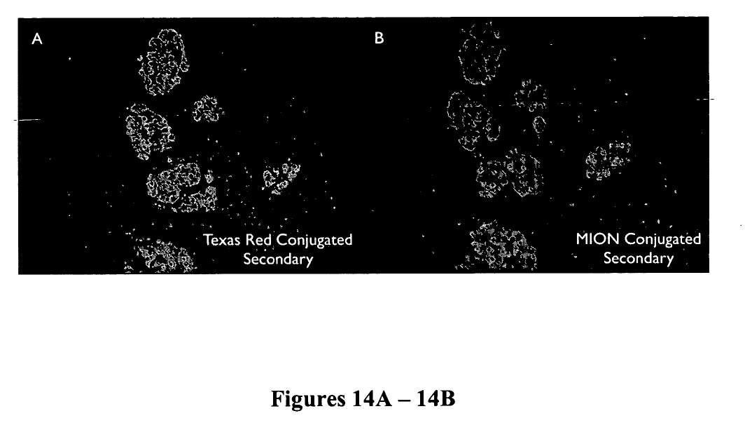

FIGS. 15A-15B. T1 weighted images of a rat pre (FIG. 15A) and post (FIG. 15B) injection of the phospholipid coated nanoparticles. Notice the darkening of the liver in the post-injection image. The nanoparticles generate a change in intensity that is easily detected in T1, T2, T2*, and proton weighted MRI scans.

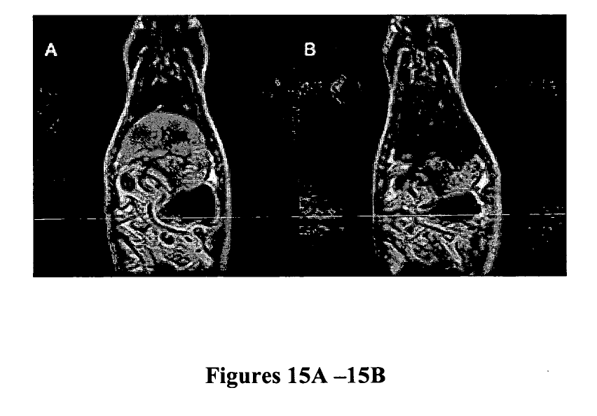

### DETAILED DESCRIPTION OF THE INVENTION

The invention provides for methods of detecting or sensing or imaging specific cells (the cell of interest) within a tissue or in vivo, or in vitro by administering to the tissue or the subject an effective amount of an iron oxide nanoparticle(s) conjugated to an antibody that is specific for the cell of interest. The invention also provides methods for creating or producing or obtaining iron oxide nanoparticles comprising injecting iron pentacarbonyl into a reaction mixture of oleic acid and trioctylamine (TOA) previously heated to 200° C. The invention also provides methods for synthesis and conjugation of iron oxide nanoparticles to antibodies for targeting specific cells using fluorescence and MR imaging techniques.

The terms “nanoparticle” and “nanocrystal” are use interchangeable herein.

The invention describes a method for the preparation of water-soluble magnetic iron oxide nanocrystals (MIONs) and conjugation of these MIONs to antibodies. The invention encompasses a new MION contrast agent that can be used for MR and fluorescence imaging of tissues.

The iron oxide nanocrystals are between 7-14 nm and are made water-soluble by encapsulation within niPEG phospholipid micelles. Phospholipids are molecules that contain long hydrophobic tail at one end, and a polar head at the other end. In a solution of chloroform, the mPEG 750 phospholipid forms micelles with the non-polar hydrophobic chains at the center of the micelle, and the polar head on the micelle surface. When MIONs are introduced into the phospholipids micelle solution, the MIONs become encapsulated as the chloroform is allowed to evaporate. Once the MIONs are encapsulated within micelles, they are water-soluble. Phospholipids of various lengths can be used to coat the MIONs, however mPEG 750 gives MIONs with the highest water-solubility so far. Nonlimiting examples of phospholipids that can be used within the context of the invention include polyethylene glycol, methoxypolyethylene glycol 2000 (mPEG 2000), mPEG 2000 maleimide, 1,2-Diacyl-sn-Glycero-3-Phosphoethanolamine-N-[Methoxy(Polyethylene glycol)-350], 1,2-Diacyl-sn-Glycero-3-Phosphoethanolamine-N-[Methoxy(Polyethylene glycol)-750], 1,2-Diacyl-sn-Glycero-3-Phosphoethanolamine-N-[Methoxy(Polyethylene glycol)-2000], 1,2-Dipalmitoyl-sn-Glycero-3-Phosphocholine, cholesterol, or any combination thereof.

To create water-soluble MIONs that can be conjugated to antibodies, or other molecular targets a small amount of a thiol-functionalized phospholipids (PTE, phosphatidylthioethanol, PTE) is used in combination with mPEG 750. In one embodiment of the method, a 1:99 ratio of PTE:mPEG is used. In another embodiment of the invention, the phospholipid micelle comprises from about 0.1% to about 10% functionalized phospholipids. In yet another embodiment, the phospholipid micelle comprises about 1% functionalized phospholipids. For maleimide MPEG 2000 and fluorescent lipids, 2-4 per MION is enough but it is possible to increase it for greater visibility for fluorescence.

The encapsulated MIONs can be made fluorescent by adding a small amount of a fluorescein labeled phospholipids to the mixture of MPEG and PTE. This allows the use of fluorescent imaging to confirm the conjugation of MIONs to cells or tissues. This method also provides for an easy process for preparation of magnetic and fluorescent imaging label. In an embodiment of the invention, the phospholipids are tagged with a fluorescent marker. In a specific embodiment, the labeled phospholipid comprises 1,2-dioleoyl-sn-glycero-3-phosphoethanolamine-N-(Lissamine Rhodamine B Sulfonyl).

The MIONs are conjugated to antibodies via a heterobifunctional crosslinker molecule, SMPT. SMPT (succinimidyloxycarbonyl-alpha-(2-pyridyldithio)toluene) is a cross-linker that contains an amino-reactive NHS ester on one end and a sulfhydryl-reactive pyridyl disulfide group on the other. The pyridyl disulfide end will react with the free thiol groups at the surface of the phospholipids micelle to form a MION-Antibody conjugate and then the NHS ester will react with amino acids on the antibody surface, such as lysine or arginine.

The invention provides methods for imaging specific cells in a body of a subject with an MRI scanner, giving a non-invasive way or methodology to examine the locations and concentrations of cells and proteins in the body. The invention also provides a method for detecting a cell of interest in a subject, the method comprising administering to the subject an effective amount of an iron oxide nanoparticle-antibody conjugate, wherein the antibody specifically binds to the cell. The invention further provides administering to the subject an effective amount of an iron oxide nanoparticle-antibody conjugate, wherein the antibody specifically binds to the polypeptide. In certain embodiments of the methods of the invention, the nanoparticle is detected by magnetic resonance imaging or fluorescence imaging.

The invention utilizes a functionalized phospholipid to attach to the antibodies via a crosslinker, rather than a dextran coating or an avidin-biotin linkage. In one embodiment of the invention, the functionalized phospholipids comprise thiol-functionalized phospholipids, amine functionalized phospholipids, or any combination thereof. In another embodiment, the amine-functionalized phospholipids comprise DSPE-PEG(2000)Carboxylic Acid, DSPE-PEG(2000)Maleimide, DSPE-PEG(2000)PDP, DSPE-PEG(2000)Amine, DSPE-PEG(2000)Biotin, or any combination thereof. In yet another embodiment, the thiol-functionalized phospholipids comprise phophatidylthioethanol (PTE). In this invention, the phospholipid coating is smaller than the dextran coating. The methods provided by the invention also require very little purification compared to coating with dextran. The lipid coating is also similar to the lipid structure of the cell membrane, therefore the lipid coating will appear less foreign to the subject's immune system.

The invention also provides for an oscillating magnetic field, the particles could be used for hyperthermia to destroy the cells or proteins they target. In addition, the invention provides for methods where drugs are attached to the phospholipid layer, resulting in a very specific drug delivery device and method.

Specific Cell Detection Using Iron Oxide Nanoparticles

The ability to noninvasively detect and image specific cells and proteins in the body would greatly advance the way physicians diagnose and treat diseases. Imagine being able to inject a small amount of contrast agent into a body, take an MRI, and know the location and concentration of specific cells with having to make a single cut. While contrast agents exist that can target tumors or specific organs, the ability to target specific cells and proteins is a new and developing technology. This invention provides a method for conjugating antibodies to γ-Fe₂O₃ nanoparticles and methods for detecting changes in the intensity of MR images due to the presence of the nanoparticles. The invention also provides methods for determining the concentration of the nanoparticles, and thus the cell or protein, based on the magnitude of the intensity change.

A goal of the invention is to be able to image beta cells in diabetic patients that had received islet cell transplants. One embodiment of the invention comprises an anti-insulin antibody conjugated to a nanoparticle. Diabetes is a disease that affects over 15 million people in the United States alone. There are two types of diabetes, type 1 and type 2. Type 1 diabetes occurs in 5 to 10% of the cases and usually appears during childhood. People with type 1 diabetes are unable to produce insulin, and must take shots to control the insulin and glucose levels in their body. On the other hand, type 2 diabetes usually appears after childhood and can usually be controlled by diet and medication, without the need for injections. Diabetes is a lifelong disease with serious complications, such as blindness, kidney failure, loss of limbs and even death. Being able to cure, or even just better control diabetes, can significantly improve the lives of people suffering from diabetes. A new method of treatment for severe cases of diabetes is an islet cell transplant. Using special enzymes, islet cells are removed from the donor pancreases and then injected in a vein near the liver. The islets attach themselves to the liver and begin to produce insulin. Current islet cell transplants can be successful for several years, but over time the body can reject the islet transplant and thus, being able to tag and image the islet cells after transplantation would allow doctors to watch the transplanted cells and follow the success of the transplant over time.

Iron Oxide Nanoparticles

Maghemite, γ-Fe₂O₃, is a reddish-brown material found in soil and magnetic pigments. It has a cubic unit cell containing 32 O ions, 211/3 FeIII ions and 21/3 vacancies, with the cations distributed over the 8 tetrahedral and 16 octahedrals sites and the valences randomly distributed only among the octahedral sites. The magnetic structure of maghemite is made up of a tetrahedral and an octahedral sublattice, where the magnetic moments of the atoms in each lattice are parallel to the atoms in their lattice and antiparallel to atoms in the other lattice. In bulk, maghemite is ferrimagnetic, however small enough nanoparticles exhibit superparamagnetism. As in the synthesis provided by this invention, maghemite is usually formed from other iron oxides, such as FeO, and adopts the structure of the precursor iron oxide. The invention provides for nanoparticles comprising maghemite.

Magnetic Resonance Imaging and Contrast Agents

Magnetic resonance imaging uses high magnetic fields and radio frequency pulses to generate extremely accurate images of the body. The magnetic field inside our MR scanner is 1.5 T. Initially, the spins of the protons in the body or sample are spinning at random in every orientation in a state of rest. Once placed in the scanner, the high magnetic field causes the protons to align their spins with the magnetic field. A radio frequency pulse is applied and is absorbed by the protons, allowing them to rotate their spins away from their alignment with the magnetic field. As the spins slowly move back towards their previous alignment, the protons release the absorbed energy in the form of another radio frequency pulse with a phase dependent on the material. The scanner picks up the radio frequency signals from the protons and places them into a grid in frequency space. The grid is then Fourier transformed from frequency space into real space, which generates an image of the material. Regions in the material where many protons were in phase will give off a strong coherent signal, while the signal from regions where the protons were out of phase will interfere with each other resulting in a very low intensity signal. When the scanner processes the signals, regions with high signal intensity are shown as white, while regions with low intensity are very dark.

The two main scan parameters are the repetition time and the echo time. The repetition time (TR) is the amount of time in between the radio frequency pulses. This determines the amount of time that the protons have to relax back to the aligned state before the next pulse. The echo time (TE) is the amount of time between the pulse and the recording of the proton signal. Different materials have different relaxation times so changing the echo time changes the window of relaxations times that is recorded by the scanner. Tissue with faster relaxation times need shorter TEs while tissue that relaxes slower will appear brighter for longer TEs.

Three types of MRI scans were used: T1, T2, and T2*. The T1 time is the longitudinal (spin lattice) relaxation time, which is the time required for 63% of the spins to return to alignment. In a T1 weighted scan, the TR is less than 1000 ms and the TE is greater than 30 ms but still short. Thus, material with a short T1 will appear bright in a T1 weighted image, while material with long T1 or T2 will appear dark. The T2 time is the transverse (spin spin) relaxation time, this is the amount of time required for the signal to decay 63% due to protons losing their transverse magnetization via energy exchanges with other protons. T2 is generally less than T1 because it takes less time to transfer energy between protons than to transfer energy between the protons and the lattice. In T2 weighted scans, the TR is greater than 1500 ms and the TE is greater than 60 ms, thus material with short T2 appear dark while material with long T2 appear bight. The T2 time is similar the T2 time but because it takes into account the inhomogeneity of the static magnetic field and spin spin relaxation in the body, the loss of phase coherence of the signal is much more rapid and thus the T2* time is always shorter than the T2 time. In addition to changing the TE and TR of the scan, other ways to select specific types of material includes altering the pulse sequences, manipulation of the magnetic gradients, changing the way the signals are placed into frequency space grid and how they are analyzed, and finally by the addition of contrast agents.

Contrast agents are injected into the body into to selectively alter the contrast of a specific structure or region and thus improve the sensitivity or specificity of the scan. The contrast agents alter the T1, T2 and T2* relaxation times which changes the signal coming from that region and thus changes how the region appears in the image. Positive contrast agents act mainly on T1 relaxation and enhancement in the signal from the region, which increases the brightness of the region in the resulting image. Negative contrast agents tend to act more on the T2 relaxation, causing a reduction in the signal that results in a dark spot in the image. There are many different types of contrast agents used with a variety of tissue targeting methods. The most common contrast agent is Gadolinium or molecules that contain a Gadolinium ion, which is used because of the large number of unpaired electrons in its outer shell. A newer contrast agent still being tested is iron oxide nanoparticles. These nanoparticles are very small and significantly less harmful than Gadolinium, which needs to be chelated to be considered safe. Iron oxide nanoparticles exhibit a superparamagnetic property which allows them to easily and rapidly change their magnetic moments and thus have a large effect on surrounding protons. They are very good negative contrast agents, as has been shown in many studies. Some of these molecules depend on the target structure, such as a tumor, to preferentially absorb most of the contrast agent while others are filtered naturally into target organs such as the spleen, the liver, and the lymph nodes. The invention provides methods to target specific cells through the use of antibodies. By coating the contrast agent with specific antibodies, the contrast agents are provided with a special tag that tells the body exactly what it wants to attach to. This method will allow one to selectively image very specific types of cells in the body which could be very beneficial for noninvasively learning about the types and concentrations of specific cells in the body.

Scanning Procedures

An agarose gel was used to prepare the γ-Fe₂O₃ nanoparticles for MRI scanning. In initial scan attempts, the phantoms were made by simply putting several dilutions of the nanoparticles in water into either 2 ml or 12 mL glass vials or 15 mL centrifuge tubes. Unfortunately, water is not viscous enough to prevent the strong magnetic field of the scanner from pulling all the particles out of solution, thus it was impossible to get a uniform intensity from the phantoms. Additionally, gelatin was used to hold the nanoparticles, but while the gelatin did prevent the nanoparticles from being pulled out of solution, it had a tendency to solidify with tiny bubbles in the gel if it was shaken too much or cooled too fast, which appeared as small black spots in the MRI scans. Thus, agarose agar was used because it resulted in clear, bubble-free scans. Dilutions of the γ-Fe₂O₃ particles in water were prepared at double the desired concentration, and then an equal volume of a 0.5% agarose agar solution was added to the vials. A typical set of dilutions used was 0.1, 0.2, 0.5, 1, 2, 5, 10, 20, 50 and 100, where 100 is the concentration by volume when the nanoparticles are 1 μm apart. The solutions were then mixed using a vortexer, and placed in a 4° C. refrigerator to solidify.

The samples were scanned using a 1.5T Phillips MR Scanner using a transmit receive head coil. Due to the small size of the samples, a scout scan was run first with a large bottle phantom in the machine. After scanning the large phantom, the scanner accepted that there was something in the scanner, and allowed running of smaller samples. When scanning a series of dilutions, the vials were placed on a tray in order of descending concentrations. Single phantom scans were simply placed near the center of the coil. There were four different types of scans performed on the samples. The first scan performed was a scout scan, this is a basic scan that was used to find the location of the vials in the scanner so the slices could be set up in the following scans. The second type of scan was a T1 spin echo (SE) scan, which was not used as often. The basic settings for the T1 scans were a TR of 100 ms, a TE of 15 ms. The two most frequently used scans were the T2 and T2* scans. The T2 scan was a TSE sequence with a TR of 3000 ms and a TE in the range of 100 to 400 ms. The T2* scan was a FFE sequence with a TR of 500 ms and a TE in the range of 10 to 50. To obtain the T2 time of the dilution samples, a CPMG scan was used. This is a SE scan with a TSE factor of 20 and 20 echoes used. The TR was 3000 ms and the TE were multiples of 20 ranging from 20 to 400 ms. The thickness of the slices was generally 1 to 2 mm, with a gap size between 0 and 2 mm. Larger, more uniform samples had thicker slices, while in small samples the slices were positioned and to give the most uniform, bubble free slice possible.

Antibody Targeting

Antibodies are highly specific proteins, produced by the immune system, that bind to and help neutralize a wide range of antigens, such as toxins, bacteria, viruses, cells and specific molecules and proteins in order to protect the body. The general structure of the antibody is in the shape of the letter Y, with two heavy chains and two light chains held together by disulfide bonds. The general length of the stem and the beginning of the arms is relatively constant, however the total length of the arms varies between different types of antibodies. At the tip of each arm is a specific antigen-binding site which determines what antigen the antibody can attach to. The stem of the antibody is specific to the particular animal that the antibody is made in. Because antibodies are very good at finding their particular target and only attaching to that target, they are commonly used in biology to stain and sort specific types of cells or proteins, this property also makes them an ideal candidate for helping our nanoparticles target specific cells in the body.

In the body, antibodies attach to their specific antigen and set off a chain reaction of effects in order to prevent damage to the body from foreign invaders. Upon attachment to its complementary antigen, the antibody can either render the antigen neutral itself or, with the assistance of other effector agents, it can repair or destroy the antigen or infected cells. When tissue is stained or antibodies are used to help nanoparticles target specific proteins, one relies on the specificity of the antibody to find its specific target. Because the stems of most antibodies share very similar chemistry and have many different types of chemical groups to bond to, attaching molecules to the stem of different antibodies without interfering with the function of the antibodies is not very difficult. This also allows provides a generic attaching procedure that can be used for a variety of antibodies.

The invention provides for attachment of the nanoparticles to the primary antibody. The invention provides methods for attaching the nanoparticle to an antibody in the presence of a crosslinking agent or in the absence of a crosslinking agent. The ratios of the crosslinking agent SMPT to antibody and antibody to nanoparticles can be optimized. The methods provide for limits on dilution during the gel filtration step, as the reaction is more successful when the solution is more concentrated. In one embodiment of the invention, the crosslinking agent comprises the crosslinking agent comprises a heterobifunctional crosslinking agent. In another embodiment, the crosslinking agent comprises SMPT (4-succinimidyloxycarbonyl-α-methyl-α-(2-pyridylditio)toluene), sulfo-LC-SMPT (sulfosuccinimidyl-6-(α-methyl-α-(2-pyridylthio)toluamido) hexanoate, Traut's reagent (2-Iminothiolane•HCl), or any combination thereof. A non-limiting exemplary method of conjugation uses maleimide functionalized MPEG2000 which links to a thiol group on the modified antibody. In another non-limiting example, Traut's reagent (2-Iminothiolane•HCl) is utilized to modify the amine groups on the antibody into thiol groups.

γ-Fe₂O₃ nanoparticles can be coated with fluorescein-tagged phospholipids and that the nanoparticles do not interact with the pancreas tissue. Using an anti-insulin primary antibody, a standard immunohistochemistry stain with pancreas tissue was used to verify that the antibodies worked and to get a general idea of what the stained islet cells should look like and how they are dispersed throughout the pancreas (Example 1). Finally, the nanoparticles can be conjugated to the Texas Red tagged secondary antibodies without interfering with the function of the antibody and can successfully stain the islet cells (Example 1).

A determination is done to determine the optimal size of the nanoparticles for maximum effect, as well as confirm the composition and purity of our samples. The concentration of the sample will be determined and related to the concentration to the change in concentration. Being able to relate the change in intensity to a specific concentration of particles will give one a more accurate measurement of the relative amount of the targeted cell in the body, rather than just showing if the cells are present or not.

There are several different types of experiments with the antibodies that are described herein. First, the conjugation of the antibodies to the nanoparticles is to be refined. The next step is showing that the nanoparticle-antibody conjugates attach to cells in a cell culture in a high enough concentration that the change in intensity can be detected. Finally, nanoparticles conjugated with MHC Class II antibodies are injected into a disease model rat and a determination is made to see if the conjugate attaches to the diseased organ and reduces the intensity of the organ in the MR image. The invention also provides for beta cell imaging, with an antibody specific for the surface of a beta cell. Alternatively, the invention provides for specific uptake of the beta cells of the nanoparticles. The invention provides for conjugation of a γ-Fe₂O₃ nanoparticle to a specific antibody, injection of the conjugate into a rat, and detection of a change in the intensity in a specific region of the MR image due to the attachment of the antibodies to the specific target.

There are several measurements to take to better characterize the nanoparticles and fine tune selection of the nanoparticles to be used for conjugation. One aspect to be tested is the critical size for superparamagnetism. It is important to know at what size the nanoparticles stop being super paramagnetic, in order to maximize the effect without the nanoparticles being split into domains. This is done by spin coating a drop of a nanoparticle solution on a silicon wafer measuring the magnetization of the nanoparticles for varying sizes of nanoparticles.

The invention also provides for a method to accurately determine the total mass of the nanoparticles and the concentrations of the samples. Because the diameter of the nanoparticles can be measured, one can calculate both using the elemental analysis procedure. This involves synthesizing a batch of nanoparticles over 0.5 g, TEMing the nanoparticles to measure the diameter, finding the absorption of the solution at varying concentrations, and then evaporating off the water to get an accurate measurement of the amounts of all the different elements in the nanoparticles. From the total mass of the iron oxide and the diameter of the nanoparticles, one can calculate the number of nanoparticles that were in the solution and then get the mass of phospholipids coating each nanoparticle. One can then plot the absorption versus the concentration and get a curve that would allow one to determine the concentration of any sample using the absorption of the sample.

The invention also provides for other measurements to be determined such as x-ray diffraction images and the spectra of the nanoparticles to confirm the nanoparticles are indeed γ-Fe₂O₃ and verify the purity of the sample.

Magnetic Resonance Imaging (MRI)

With accurate determination of the concentration of the solutions, a relationship between the change in contrast and the concentration of the iron oxide nanoparticles in the sample is identified. Some initial scans have completed. The invention provides for further dilutions and a wider range of sizes to give further information about the relationship between the concentration and the contrast and how it is affected by the size of the nanoparticles. While a majority of the effects seem to be for concentrations over about 10 MIONs/μm³, using concentrations between about 0.1 and about 200 MIONs/μm³ would give a more complete idea of the behavior of the curve.

Another method provided by then invention is to vary the amount of agarose agar in the gel to determine how the density of the gel affects the relationship between the concentration and the contrast. For this experiment, the agarose solution may be varied from about 0.5% to about 5% agarose by weight to simulate varying densities of tissue.

Antibody and Tissue Experiments

Initial conjugation of the nanoparticles to the secondary antibodies was successful. The invention provides using unconjugated secondary antibodies for the conjugation step. Without the Texas Red tags, the secondary antibodies will have more space for the linker molecules to attach. Using secondary antibodies rather than primaries will also give a stronger signal because multiple secondary antibodies can attach the to primary antibody and multiply the resulting signal, while only one primary antibody can attach to each antigen and thus the signal is weaker.

The invention provides varying the ratios of the link molecule, SMPT, to antibody to ensure that the reaction is done with an excess of linker molecules but without causing the solution to aggregate due to excess linker interactions. The invention provides using a different linker molecule, such as Sulfo-LC-SMPT, which is similar to SMPT but has a longer spacer arm and is soluble in water, which may allow for better attachment to the antibody.

The ratio of nanoparticle to antibody is to be optimized. The invention provides use of an excess of nanoparticles in the reaction to ensure that every linker molecule has a nanoparticle attached to it. This would be accomplished by increasing the amount of nanoparticles used until the intensity of the signal either plateaus or decreases.

The invention provides methods for conjugation of the nanoparticle to the antibody comprising concentrating the antibody-SMPT solution before adding the nanoparticles. This is an improvement over other methods. The higher concentration means there are more interactions between the SMPT molecules and the nanoparticles, resulting in each SMPT molecule finding a nanoparticle to conjugate. One method for increasing the concentration of the solution after filtration through the gel is to collect the filtrate into multiple small containers rather than one large container. Then, using Bradford Reagent, one can compare the protein concentrations of the samples and only combine the samples with the highest concentrations. This eliminates the extra buffer that exits the gel before and after the antibodies, thus reducing the overall volume of the sample while still collecting a majority of the antibodies. Another method for concentrating the solution involves using special centrifuge tubes. The tubes have a membrane insert with specific sized pores than lets through molecules below a specific molecular weight. Thus, the excess buffer solution flows through the membrane and into the bottom of the tube while the large antibody-SMPT conjugates are too big to pass through the membrane and stay in the insert.

MHC Class I and Class II Experiments

The first part of the cell culture experiments involves using an antibody called MHC Class I, which is a generic antibody expressed by almost every cell in the body. Cell cultures of normal human cells in varying concentrations will be washed with MHC Class I conjugated nanoparticles and then scanned in the 1.5 T MRI scanner. This method determines if the conjugate will move through the solution and attach to its target, as well as shows what concentration of cells is needed to detect the presence of the nanoparticles.

The invention includes use of MHC Class II antibodies. These antibodies are expressed by cells in distress. Thus, the antibody will not attach to normal cells unless the cell is in trouble. For the MHC Class II experiments, one can wash normal and inflamed cell cultures with the conjugates and compare the contrasts.

After demonstrating that the nanoparticle-antibody conjugates attach to the cells in the cell culture in a concentration high enough to be detected by the MRI scanner, the conjugate is used in vivo using mouse or rat disease models. Here, a mouse or rat with a particular disease is imaged on an MRI scanner, injected with the nanoparticles conjugated to MHC Class II, and then scanned again. Comparing the before and after images, a negative change in the contrast in the organ affected by the particular disease model is seen. In these methods, the amount of the nanoparticles that make it to the target organ is measured, as well as how much of the conjugate is taken up by other organs. The invention also provides for methods to determine how much of the conjugate needs to be injected into the animal for the change in contrast of the target organ to be easily detected. This can be accomplished by injecting a range of amounts and comparing the resulting contrast changes of the target organ.

Beta Cell Experiments

The invention provides for using antibody targets that stay within the cell. There have been some targets located on the membrane of the beta cell and antibodies to those targets can be conjugated to the nanoparticles.

Less specific antibodies can also be used, and in this case there is the added step of incubating the conjugate with the cells prior to transplantation. While the antibodies are not specific, attaching them prior to injecting prevents them from attaching to other types of cells.

Another solution is to get the nanoparticles into the cells either by targeting an internal beta cell protein or by having the cells uptake the nanoparticles from the medium.

The invention provides methods of synthesis and characterization to examine the properties of the nanoparticles in the MRI scanner to conjugation with antibodies and attaching to tissue and cell cultures. The invention provides a critical size for superparamagnetic γ-Fe₂O₃ nanoparticles at room temperature and methods to be able to determine the concentration of the nanoparticles solution. The relationship between the change in contrast and the concentration of the nanoparticles is determined as part of this invention, as well as the effects of nanoparticle size and tissue density.

The ability to non-invasively image and measure the concentrations of specific cell and protein types in the body would greatly improve the way physicians diagnose and treat many diseases. The invention provides for conjugation of γ-Fe₂O₃ nanoparticles to specific antibodies that attach to specific cells or proteins in the body in a concentration that allows them to be detected in MR images.

Using the methods provided by the invention, one can successfully synthesize uniform, monodisperse γ-Fe₂O₃ nanoparticles and coat them with a layer of phospholipids so they can be dispersed in water. With the addition of PTE, thiol group is added on the nanoparticle that can be used to attach an antibody linker molecule. Adding a Fluorescein-tagged phospholipid makes the nanoparticle fluoresce green, which gives one the ability to detect the presence of the nanoparticle without an MRI scan. Initial antibody experiments showed that γ-Fe₂O₃, nanoparticles can be conjugated to Texas Red tagged secondary antibodies in a concentration that could easily be visually detected in an immunohistochemistry stain.

The relationship between the concentration of nanoparticles in the material and the change in the intensity of the image are important considerations. One can then use cell cultures to determine how well the nanoparticles attach to the cells and the concentration of cells needed to be detected in MR scans. Finally, using rat disease models, in vivo testing is used to determine if one can detect the particles attaching to a particular region of the animal. The methods of the invention can be used to image beta cells with suitable antibody for binding to the surface of the beta cell.

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention.

### EXAMPLES

### Example 1

### Synthesis and Characterization of Water-Soluble Maghemite, •-Fe₂O₃ Nanoparticles

Synthesis of Maghemite, γ-Fe₂O₃ Nanoparticles

The iron oxide particles were synthesized using a method slightly modified from the Hyeon method. A solution of 3 mL (9.45 mmols) oleic acid (OA) and 15 mL (34.31 mmols) trioctylamine (TOA) was heated to 320° C. under an atmosphere of nitrogen. Once the solution reached a temperature of 200° C., 0.4 mL (3.04 mmols) iron pentacarbonyl (Fe(CO)₅) from Sigma Aldrich was injected. As the solution was heated at 320° C. for 1 hr, the color of the solution changed from its initial postinjection color of orange to clear as the iron pentacarbonyl decomposed to Fe ions. The solution then turned to an opaque black as partially oxidized Fe/FeO nanoparticles formed. During a successful synthesis, the change from clear to a translucent brown is gradual, followed by a rapid change to opaque black. After 1 hr, the solution was cooled to below 60° C. and then 0.7 g (9.32 mmols) of dehydrated trimethylamine-N-oxide (TMAO) was added and the solution was heated at 130° C. for 2 hours, and then at 320° C. for 1 hour. The iron oxide solution changed from black to a reddish-brown color after the addition of the oxidizer and then back to black/dark brown as the nanoparticles oxidized. The solution of γ-Fe2O3 was then cooled to below 40° C. and washed using chloroform and ethanol. The maghemite nanoparticles were characterized using transmission electron microscopy (Jeol CX100) with an accelerating voltage of 100 kV.

Transferring γ-Fe₂O₃, Nanoparticles to Water and Characterization of Water-soluble Nanoparticles

The γ-Fe₂O₃ nanoparticles were made water-soluble by encapsulating them within phospholipid micelles. The method used to coat the γ-Fe₂O₃ nanoparticles with phospholipids was developed by Dubertret. The chloroform in the sample solution was allowed to evaporate overnight until the sample was almost completely dry. The sample was then massed to determine the total mass of nanoparticles, and then using the mass and the diameter of the nanoparticle measured from the TEM images, the approximate number of nanoparticles was calculated. Using a 99:1 molar ratio, 0.0057 g (0.0037 mmols) of mPEG 750 PE (1,2-Distearoyl-sn-Glycero-3-Phosphoethanolamine- N-[Methoxy(Polyethylene glycol)-750]) and 0.0003 g (0.0004 mmols) PTE (1,2-Dipalmitoyl-sn-Glycero-3-Phosphothioethanol) (for example, from Genzyme and Avanti Polar Lipids) per 10 mg of γ-Fe₂O₃ were dispersed in 400 μL chloroform and allowed to sit for at least 1 hr. 20.0 μL 1,2-Dioleoylsn- Glycero-3-Phosphoethanolamine-N-(Carboxyfluorescein) (for example, from Avanti Polar Lipids) was added to the phospholipid solution for the green Fluorescein-tagged γ-Fe₂O₃ nanoparticles. The phospholipid solution was sonicated for 3 min. and then added to the dried γ-Fe₂O₃ nanoparticles. The chloroform was then allowed to evaporate until the sample was dry. Next, the dried sample was heated to 80° C. and 80° C. water was added. The solution was then sonicated and vortexed to disperse the phospholipid-coated nanoparticles into the water. The solution is then ultracentrifuged at a speed 27,000 rpm and a temperature of 7° C. for 2 hours to remove the excess phospholipids from the phospholipid coated γ-Fe₂O₃ nanoparticles. While the ability of the particle to be dispersed in water is a good indication that the particle is coated with phospholipids, the particles were also characterized using TEM images. A small amount of nanoparticles is diluted with water and then a drop of the solution is placed on the 400 copper mesh grid. The sample is then dried in a vacuum chamber overnight. The grid is then stained with a drop of 1% phosphotungstic acid, which makes the phospholipid coating visible by TEM. It also allows visualization and measurement the lipid coating around the γ-Fe₂O₃ nanoparticles.

Characterization of the maghemite nanoparticles was done using transmission electron microscopy (Jeol CX100) with an accelerating voltage of 100 kV. The uncoated γ-Fe₂O₃ samples were prepared for TEM by first making a dilute solution of γ-Fe₂O₃ nanoparticles in chloroform and then placing one drop of the dilution on a 400 mesh copper grid with Formvar backing. The samples were then dried in a vacuum chamber for 30 minutes. The water-soluble nanoparticles were prepared by making a dilute solution of γ-Fe₂O₃ nanoparticles and water. A small drop of the solution was placed on the copper grid and then the sample is placed in a vacuum chamber to dry overnight. The grid was the stained with a drop of 1% phosphotungstic acid, which stains everything but the phospholipids. This made the phospholipids visible by TEM, and allowed visualization of the uniformity of the phospholipid coating and measurement of the thickness of the coating around the γ-Fe₂O₃ nanoparticles.

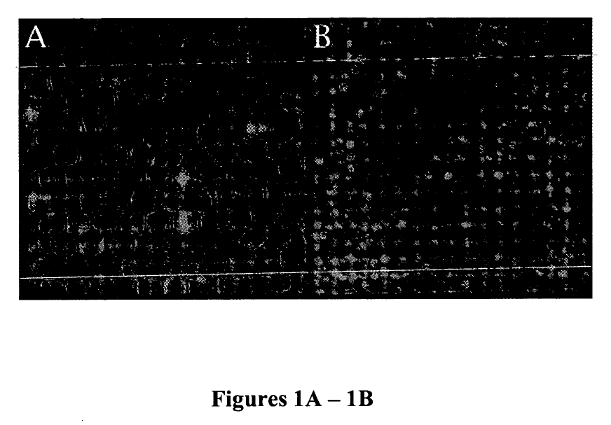

The synthesis method used was capable of producing highly uniform, monodisperse γ-Fe₂O₃ nanoparticles. FIG. 1 shows two examples of γ-Fe₂O₃ nanoparticles produced.

Injection of the iron pentacarbonyl into the oleic acid and TOA once the mixture had been heated to 200° C. had a significant effect on both the uniformity and size range of the nanoparticles, resulting in much more uniform, monodisperse nanoparticles. Injecting the iron pentac-arbonyl causes the all of the iron to decompose and begin to form particles at the same time, this is believed to be the reason for the improvement in the quality of the particles.

The nanoparticles seemed to get progressively smaller over time, going from a very uniform, monodisperse sample with a diameter around 15 nm to less uniform nanoparticles with sizes around 5 to 6 nm or not even producing nanoparticles at all. Adding more oleic acid increased the size of the nanoparticles slightly but the nanoparticles were still not as symmetrical or uniform as before. Usually, there would be a small dispersion of larger nanoparticles in a sea of small 5 to 6 nm nanoparticles. After testing all possible variables, the decomposition of the iron pentacarbonyl appeared to be the cause of the decreasing size and uniformity of the γ-Fe₂O₃ nanoparticles. Some variation may be seen between two samples made side by side on identical setups using identical procedures. No explanation for that problem has been found.

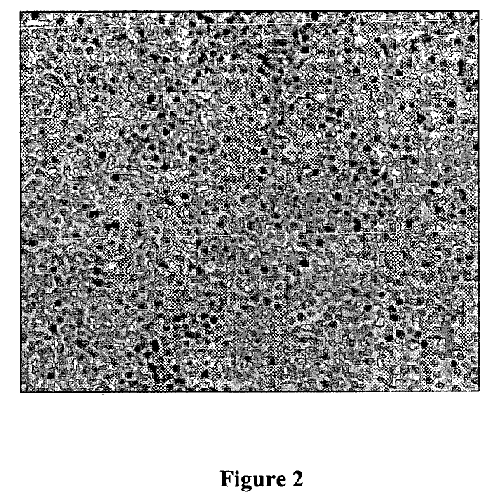

Once uniform, monodisperse samples of γ-Fe₂O₃ nanoparticles have been made, they need to be coated with a layer of phospholipids so that the nanoparticles will be soluble in water. The first samples were coated using a 60:40 molar ratio of DPPC to mPEG2000 phospholipids. While this seemed to work well with the smaller nanoparticles, 3 to 6 nm, this combination of phospholipids did not work for the larger particles. While it seemed that any ratio of DPPC to mPEG2000 would work for small nanoparticles, larger nanoparticles went into water increasingly better as the amount of mPEG to DPPC was increased. Using only mPEG2000, one may coat the whole range of sizes and disperse the particles in water with little to no nanoparticle residue left on the vial. FIG. 2 shows a sample of 4 nm nanoparticles coated with the 60:40 mixture of phospholipids in a water solution.

Reactions that resulted in very uniform nanoparticles tended to show a rapid change from the initial orange to clear and also when transitioning from clear to black. The most uniform samples exhibited a hexagonal shape. The phospholipid layer around the nanoparticles. Attaching the γ-Fe₂O₃ nanoparticles to the insulin beta cell antibodies. The antibodies are attached to the γ-Fe₂O₃ nanoparticles using a thiol functionalized phospholipid (PTE) and a heterobifunctional crosslinker molecule, SMPT. Initial experiments using a 4:1 molar ratio of mPEG750 to PTE resulted in aggregation of the particles as shown. The presence of many thiol groups on the surface of the phospholipid-coated nanocrystals could lead to significant aggregation among the particles caused by the formation of disulfide bonds between two MIONs. When the amount of PTE is reduced to 1% of the phospholipids, the particles are coated uniformly and little aggregation is observed.

Using the methods of the invention, one can synthesize uniform, monodisperse γ-Fe₂O₃ nanoparticles in a range of sizes. While the nanoparticle size can be increased by adding additional oleic acid, one has only minimal control over the size of the resulting nanoparticles. The decomposition of the iron pentacarbonyl significantly changes the size and uniformity of the particles, and adding the iron via injection significantly improves the uniformity. One can transfer the nanoparticles from a choloroform solution to water without aggregation by coating the nanoparticles with 99% mPEG750 and 1% PTE phospholipids.

The uniformity of both the size of the nanoparticles and of the phospholipid coating was confirmed using TEM imaging. These methods show that injecting the iron pentacarbonyl at 200° C., rather than adding the iron at the beginning, resulted in cleaner, more uniform nanoparticles.

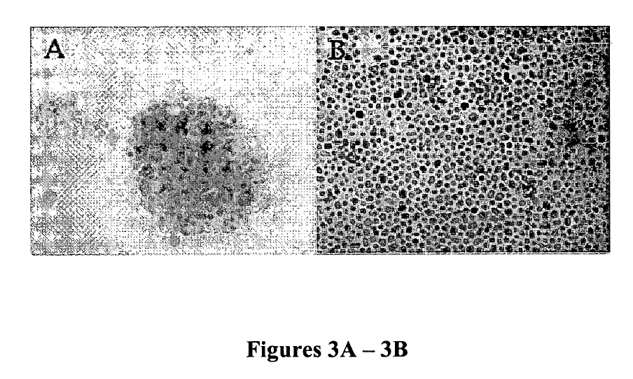

Once the nanoparticles are coated with phospholipids and dispersed in water, the invention provides a method for attaching the antibodies to the nanoparticles. The suggested linker molecule for attaching the nanoparticle to the antibody was designed to link via a disulfide bond to the conjugated particle, for example, PTE phospholipid with its thiol functional group at the end. Because of the length of PTE, smaller mPEG were used to prevent the niPEG from overtaking the PTE molecules and to give the linker molecule a better chance at attaching to the PTE. After trying a range of MPEG molecules, it was found that mPEG750 was able to successfully coat the nanoparticles while still allowing the linkers access to the PTE molecules. Initially, an 80:20 ratio of mPEG750 to PTE was used, but the nanoparticles aggregated in the solution due to linking between the thiol groups on the nanoparticles. As shown in FIG. 3, reducing the amount of PTE to 1% of the phospholipids solved the aggregation problem.

Attaching the Antibodies to the γ-Fe₂O₃ Nanoparticles

The antibodies are attached to the γ-Fe₂O₃ nanoparticles with the help of the PTE phospholipid and SMPT (4-Succinimidyloxycaroryl-α-methyl-α(2-pyridylditio)toluene), a heterobifunctional crosslinking agent. A solution of 0.4 mg (1 μmol) of SMPT was dissolved in 1 mL of acetonitrile and was added to a 500 μL solution of Texas Red tagged IgG antibodies (2 mg/mL concentration). For secondary antibody attachment, Texas Red tagged Guinea pig IgG antibodies (2 mg/mL concentration) (for example, from Abcam) were used. The conjugation to primary antibodies used Guinea pig polyclonal insulin antibodies (1 mg/mL concentration), also from Abcam. This mixture was covered with foil and allowed to stir at 4° C. in the absence of light for four hours. Gel filtration was performed using Sephadexo® G-25 to remove excess crosslinker. A slurry of Sephadexe was made with a borate buffer of pH 9 and this buffer was used as the eluant for the filtration. After filtration, 5 mg of Fluorescein tagged phospholipid coated γ-Fe₂O₃ nanoparticles was added to the Ab-SMPT conjugates and stirred for 2 hours. All procedures were performed in a refrigerated room at 4° C. and all procedures involving the Texas red-tagged secondary antibodies were performed in the absence of light. Ten liters of a borate buffer solution of phospholipid-coated nanocrystals (0.34 g in lmL buffer) was added to the filtered solution of the Ab-SMPT conjugates and stirred for 2 hours. The mixture was then ultracentrifuged at 4° C. for five hours at a speed of 27,000 rpm. The supernatant was discarded and the remaining pellet was dispersed in borate buffer.

To verify that the iron oxide nanoparticles were conjugated to the antibodies, one can do an immunohistochemistry stain using green Fluorescein-tagged nanoparticles and Texas Red-tagged secondary antibodies. This showed a positive control, stained using regular Texas Red-tagged antibodies. The islet cells are very easily distinguished from the background. The slides were prepared as described herein with the nanoparticle conjugated antibodies, only skipping the primary antibody. The nanoparticles do not stick to the tissue and a faint trace of green is only observed in cracks in the tissue and not on the tissue itself.

Tissue Preparation, Immunohistochemistry and Imaging

The visual imaging of the nanoparticle-antibody conjugates was done using 10% neutral formalin fixed human pancreas tissue in paraffin procured from Maxim Biotech, Inc. The paraffin blocks of tissue were sliced into 5 μm thick sections and placed on glass slides by the Histology Lab. The tissue sections were deparaffinized in xylene and then rehydrated using 100%, 90%, 75%, and 50% ethanol to buffered phosphate solution. All steps were performed manually at room temperature. Antigen retrieval was performed by immersing the slides in proteinase K (1:1000 dilution) for 15 minutes. Proteinase K is an enzyme that unmasks the antigens and results in a better stain of the tissue. After antigen retrieval, the slides were washed 3 times for 3 minutes each in a cold PBS/0.1% Brij-35 solution. Next, the endogenous peroxidase activity was quenched by submerging the slides in a 3% hydrogen peroxide in methanol solution for 20 minutes. The blocking of endogenous peroxidase activity in the tissue prevents the appearance of high, nonspecific background staining on the slide due to interactions between the peroxidase and the antibodies. The slides were then rinsed once in PBS for 3 minutes and incubated with avidin and biotin from Vector Labs for 30 minutes each with a 3-minute rinse in PBS in-between them. The avidin-biotin step prevents nonspecific binding between the avidin or biotin and the reagents, which can cause a diff-use weak signal throughout the tissue. Nonspecific binding sites on the tissue was then blocked by incubating in CAS for 60 minutes. The Insulin primary antibody (1:100 dilution; Abcam, Cambridge, Mass.) was incubated on the tissue for 60 minutes, followed by 2 rinses in PBS, and then the section were incubated with the Texas Red conjugated secondary antibody (1:100 dilution; Abcam, Cambridge, Mass.). Finally, the slides were rinsed twice in PBS and mounted with a DAPI-containing medium. The DAPI stains the nucleus of the cells fluorescent blue, allowing one to see all the cells in the tissue, rather than just the islets against a black background. This helps with identifying structures and positions of islets in the tissue, as well as producing a more complete image. All steps in the above procedure were completed manually and at room temperature.

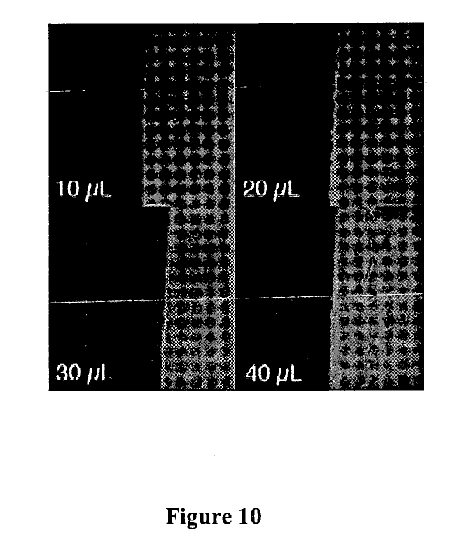

The first fluorescence experiment was designed to determine if the γ-Fe₂O₃ nanoparticles could be made to fluoresce green. For this experiment 10, 20, 30, or 40 μL of the green Fluorescein conjugated phospholipids was added to the mPEG750 and PTE coating solution. The nanoparticles dispersed very well into water following the coating process. A small drop of each solution was placed on a slide and the fluorescence of each slide was examined. As shown in FIG. 10, there is no significant difference between the intensity of the green fluorescence for the different amounts of the Fluorescein-tagged phospholipid added.

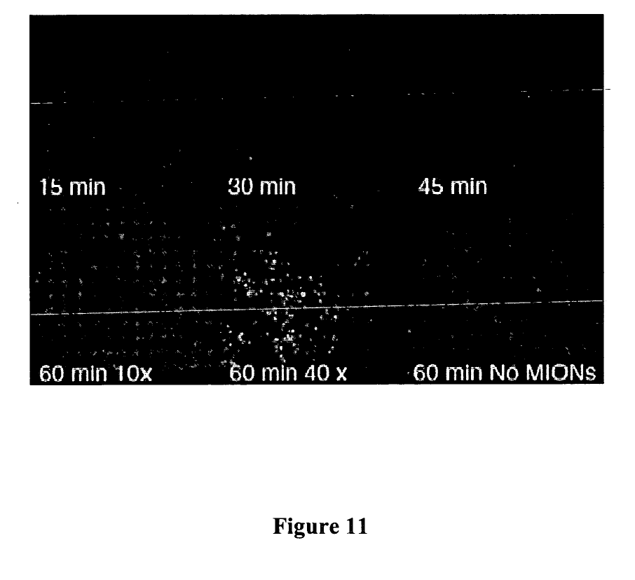

The pancreas tissue was then washed with a solution of the Fluorescein-tagged nanoparticles to determine if there was any significant interaction between the nanoparticles and the tissue, prior to the addition of the antibodies. FIG. 11 shows images of pancreas tissue after being incubated with a 100 μL of Fluorescein-tagged nanoparticles for 15, 30, 45 and 60 minutes. As shown in FIG. 11, there is a lack of green spots or green tinge on the tissue which would indicate the presence of the nanoparticles. Even after allowing the nanoparticle solution to incubate on the tissue for an hour, no evidence has been found of the nanoparticles on the tissue after the standard rinse procedure. The only green tinged areas on the slides were in the cracks between the tissue, where the nanoparticles probably got stuck or trapped and were not completely rinsed away. In conclusion, the γ-Fe₂O₃ nanoparticles do not interact with the tissue and so any attachment of the nanoparticles after conjugation to the antibodies would be due to the antibodies and not the nanoparticles.

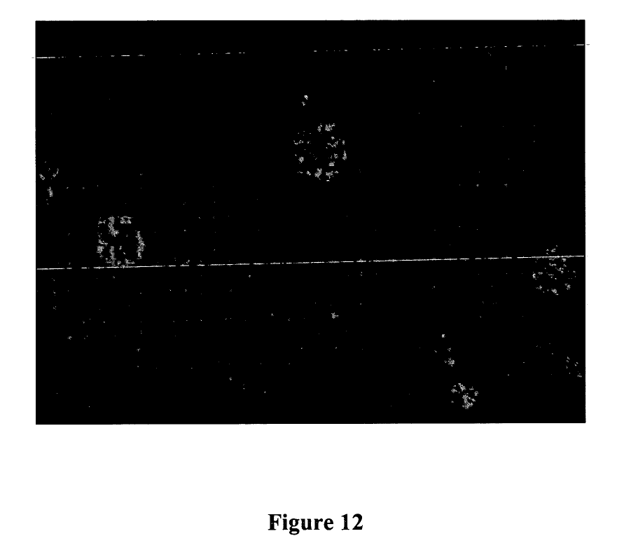

The next experiment was a basic immunohistochemistry stain for beta cells in the pancreas tissue. The purpose of this experiment was two-fold, first to check that the antibodies worked and to get a rough idea of the dilutions that would be needed, and secondly, to get a visual example of what was expected once the tissue was stained with the Fluorescein conjugated nanoparticles. FIG. 12 shows the pancreas tissue stained with an Anti-Insulin primary and a Texas Red conjugated secondary. The islet cells stand out very well from the rest of the tissue and there is very little background fluorescence. The primary antibody for this stain is very specific and resulted in a very intense, clean stain of the beta cells.

Pancreas tissue stained with the nanoparticle antibody conjugation was done. The position of the Texas Red and green Fluorescein tags are the same, which allows one to conclude that the iron oxide nanoparticles must be conjugated to the antibodies. Because the strain still works, one can also conclude that the method of attachment does not interfere with the specificity or functioning of the antibody.

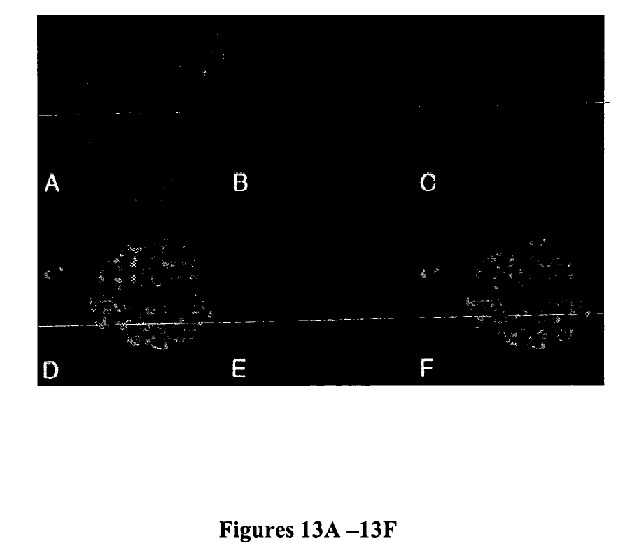

Once it had been shown that green fluorescent nanoparticles can be created and that a specific working antibody and staining procedure had been developed, the nanoparticles were conjugated to the secondary antibody. The purpose of red and green fluorescence molecules was to allow one to see where the antibodies and the nanoparticles were. If the antibodies and nanoparticles were conjugated, one would expect to see yellow spots as a result of the overlapping red and green fluorescence. The results of the conjugation between the Fluorescein-tagged nanoparticles and the Texas Red tagged secondary antibodies are shown in FIG. 13.

The top images in FIG. 13 are from the stain using the Fluorescein-tagged nanoparticles conjugated to the Texas Red-tagged secondary antibodies, while the bottom images are from a stain using Texas Red-tagged secondary antibodies. FIGS. 13A and 13D show the stains as they appear in full color, notice how intense the red color is in FIG. 13D, while in FIG. 13A the islet is a pale pink. The red channel was removed in FIGS. 13B and 13E, and the green channel was removed in FIGS. 13C and 13F. When the red channel is removed, the islet cell in FIG. 13E does not show up at all, however in FIG. 13B, the islet cell is still seen but it is now green. The green fluorescence from the nanoparticles in FIG. 13B is identical to the red fluorescence from the Texas Red secondary antibodies in FIG. 13C, which implies that they are conjugated and that they conjugation does not interfere with the function of the antibody. The green fluorescence is not as bright as the Texas Red, but this is reasonable as they are different molecules and the antibody already had four to five Texas Red molecules attached before attaching it to the nanoparticles so there were less bonding site available for attachment.

Magnetic Resonance (MR) Imaging Preparation and Scanning

The γ-Fe₂O₃-antibody conjugate was prepared for MR imaging by first preparing a slide of human pancreas tissue as shown above, and then staining the tissue using γ-Fe₂O₃ nanoparticles conjugated to the primary antibody. The finished slides were examined using a fluorescence microscope, to verify the specific staining of the tissue. A thin layer of 5% agarose agar solution, approximately 0.5 cm deep, was added to a small tray and allowed to set. Once the agar was set, the stained slide was placed in the center of the tray and another thin layer of the agar solution was added and allowed to set. The sample was then scanned using a 1.5T Phillips MR Scanner using the transmit receive head coil. Two types of MR images are taken, a T2 SE scan with a TR of 3000 ms, a TE of 200 ms, and a 2.0 mm slice thickness and a T2* FFE scan with a TR of 500 ms, a TE of 20 ms, and a 2.0 mm slice thickness. Intensity levels of the resulting images were measured using OsiriX software. This software allows one to measure the chance in the intensity of the image over a series of scans. By plotting the intensity versus the TE time, one can fit an exponential to the curve and get the T2 decay time for the particles. Then plotting the T2 times versus the concentration of the iron in the dilutions, one can determine the relationship between the change in the intensity of the image and the concentration of iron.

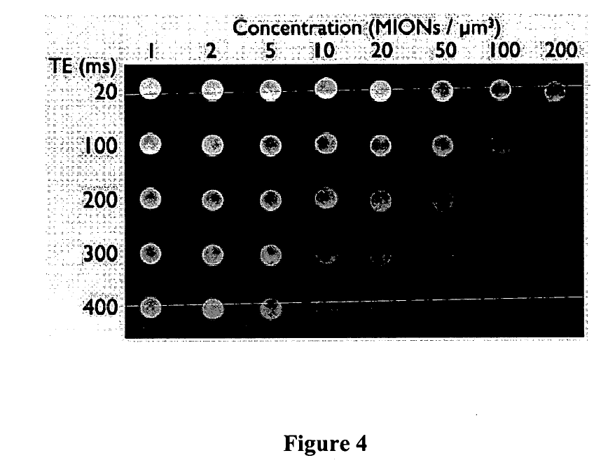

Initial MR scans were to determine if the γ-Fe₂O₃ nanoparticles could be detected in the scanner, while future scans were taken to figure out if the concentration of the nanoparticles could be determined based on the change in contrast of the image. Once uniform γ-Fe₂O₃ nanoparticle solutions were made in gelatin, a series of dilutions were made in gelatin. The dilutions were made by progressively diluting the solution each time so that, while the exact concentration of the solution was unknown, the relationship between the dilutions was correct. FIG. 4 shows images from a CPMG scan of a series of dilutions of 4 nm γ-Fe₂O₃ nanoparticles in gelatin over a range of TE times. As the concentration of the sample increases from 1 MION/μm³ to 200 MIONs/μm³, the change in the intensity of the signal is very noticeable. Visually, a noticeable difference can be seen in the intensity by the time 10 MIONs/μm³ is reached.

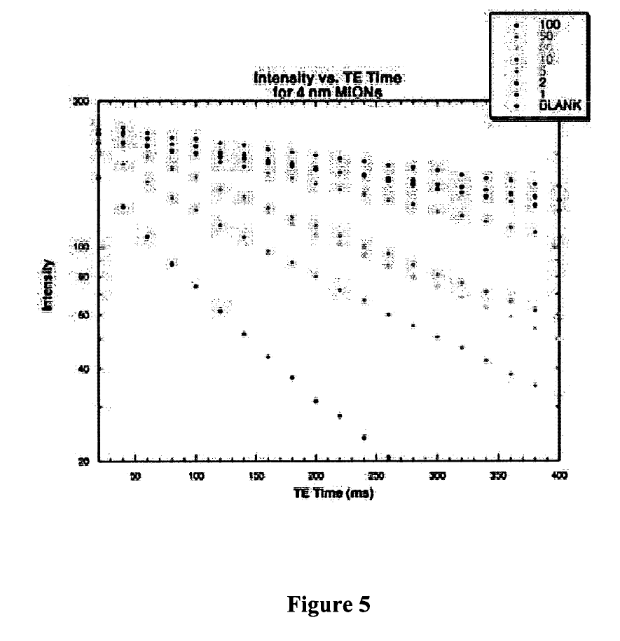

Plotting the intensity versus the TE shows that the intensity is related exponentially to the TE time and is inversely related to the concentrations of the dilutions, as seen in FIG. 5. The slope of the lines is the negative of the R2 values for the dilutions. Thus, one can see that the R2 values increase with the concentration of MIONs.

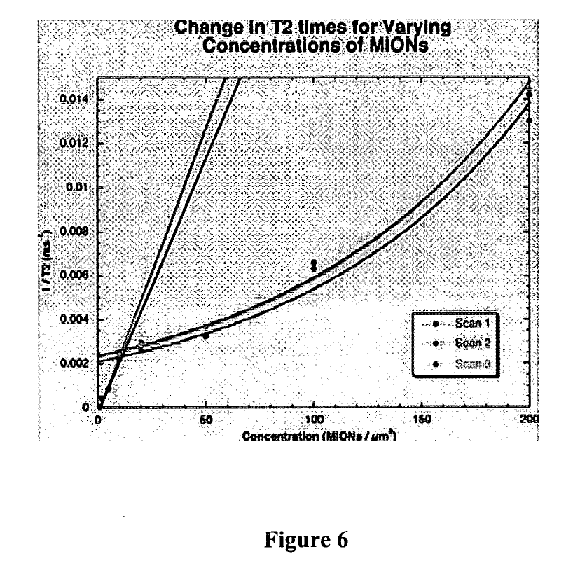

Plotting the R2 values versus the concentration for the 4 nm dilutions in the above plot gives the relationship between the relaxation rate and the concentration of γ-Fe₂O₃ nanoparticles in the dilutions. In FIG. 6, the relationship seems linear up to a concentration of about 10 MIONs/μm³, where the curve begins to grow exponentially with time.

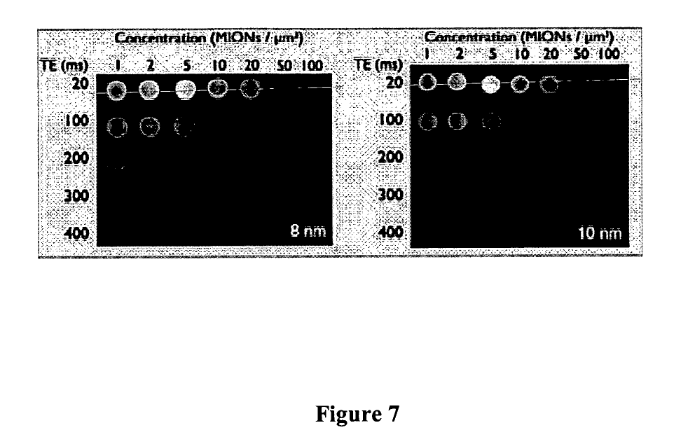

Our next experiments involved repeating the CPMG scans on samples of larger particles to determine how the change in size effected the change in intensity. These scans were done using 8 nm and 10 nm γ-Fe₂O₃ nanoparticles, although this time the samples were in an agarose gel rather than gelatin. FIG. 7 shows the MRI scans of the samples for various concentrations and TE times. The samples exhibit the same behavior as seen in the 4 nm samples, decreasing in intensity as the concentration or TE time is increased, although notice that these samples show significant decreases in intensity for all concentrations, not just samples above 10 MIONs/μm³. However, for concentrations above 10 MIONs/μm³, the spots are essentially completely knocked out for TE times of 100 ms and greater.

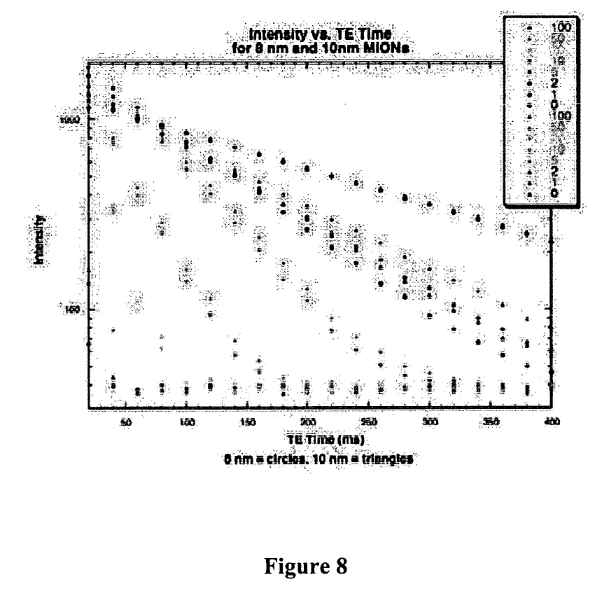

Looking at plots of the intensity versus the TE time for each sample, one notices that while it looks similar to the plot for the 4 nm samples, the drop in intensity for the higher concentrations it much more noticeable. As shown in FIG. 8, for dilutions with concentrations above 20 MIONs/μm³, the intensity drops off right away. Additionally, the curves are very similar despite the different sizes of the particles.

One concern is that the dilutions were made based on overall mass of nanoparticles and not by number of nanoparticle, thus the effect may be more a result of the total amount of γ-Fe₂O₃ in the sample rather than the number of nanoparticles. Another consideration is the critical size of the nanoparticles, when they separate into domains and lose their superparamagnetic property, which might have a significant effect on the range or effectiveness of the nanoparticles in reducing the water signal.

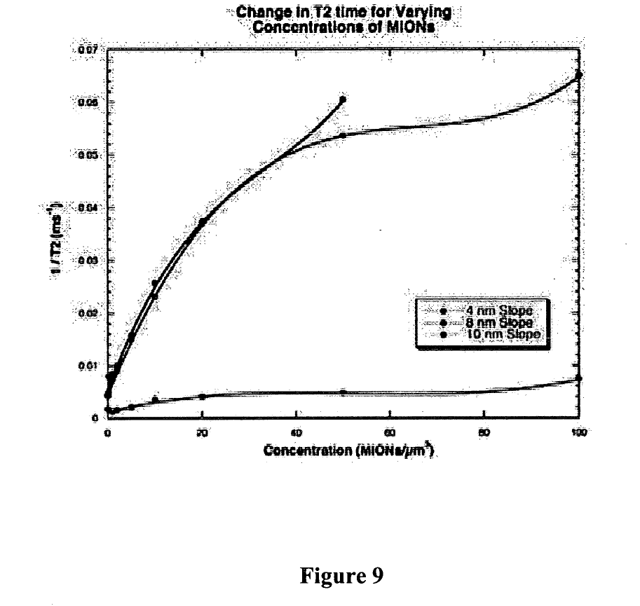

If the relaxation rate is compared with the concentration of the three different dilution sets, all three curves have the same basic shape, as shown in FIG. 9. However, the magnitude is significantly higher for the 8 and 10 nm nanoparticles compared to the 4 nm nanoparticles. This could be a result of the larger samples being suspended in the agarose agar rather then the gelatin, and is something that should be examined further.

The curve fit in the FIG. 9 is a third order polynomial, which is not what was expected but appears to fit the data well. An exponential or biexponential fit was expected, which is still possible as a more refined fitting program is used and the problem is examined further.

Thus, the inventive γ-Fe₂O₃ nanoparticles are able to be detected in the MRI scanner and the contrast can be controlled by altering the concentration of the nanoparticles in the sample. The intensity of the image decreases as the concentration of the nanoparticles increases or as the TE time increases. The 4 nm particles showed obvious contrast changes for concentrations of 10 MIONs/μm³ and above, while the larger nanoparticles showed obvious contrast changes for all concentrations. For higher concentrations and TE times in the larger particles, the intensity dropped off quickly and the spots were indistinguishable from the black background. The relationship between the relaxation rate and the concentration of γ-Fe₂O₃ nanoparticles in the solution is more complicated than a simple linear or exponential fit and has not yet been determined.

### Example 2

### Preparation of Antibody Conjugated, Phospholipid Coated •-Fe₂O₃ Iron Oxide Nanoparticles

Synthesis of the γ-Fe₂O₃ Nanoparticles

The nanoparticles are synthesized from an iron pentacarbonyl precursor using the Hyeon method. 15 Ml trioctylamine (34.31 mmol) and 3 Ml oleic acid (9.45 mmol) are heated to about 200° C. under an atmosphere of nitrogen. Once the solution has leveled off at 200° C., 0.4 Ml Fe(CO)₅ (3.04 mmol) is injected and heated to reflux (about 310° C.). Nucleation occurs during heating between about 310° C. and about 330° C. As the iron pentacarbonyl decomposes into Fe ions, the solution transforms from a transparent, bright orange to clear. After approximately 1 hr. of heating, the solution rapidly turns from clear to an opaque black indicating the start of nucleation of Fe/FeO nanoparticles. Once the nucleation begins, one continues heating the solution for 5 to 15 minutes depending on the size of nanoparticles desired. The solution is then cooled to 130° C. and 0.7 g dehydrated trimethylamine-N-oxide (9.32 mmol) is added. The solution is heated at 130° C. for 2 hrs. then heat to reflux for another hour (310° C.). The iron oxide solution turns to a reddish-brown color following the addition of the oxidizer, and then to a dark brown as the nanoparticles are oxidized. Finally, the solution is cooled to room temperature, precipitated from the host solvent using ethanol and hexane, and redispersed into hexane.

Prior to coating any nanoparticles, all samples are examined using transmission electron microscopy (TEM) to check the size and uniformity of the sample. A typical synthesis results in highly uniform nanoparticles with a diameter of approximately 5 nm with a standard deviation of less than 5%.

Coating the γ-Fe₂O₃ Nanovarticles

A coating of phospholipids is used to render the nanoparticles water-soluble. The nanoparticles are precipitated in methanol and centrifuged at 13,400 rpm for 2 minutes. After centrifugation, the supernatant is removed and the precipitated nanoparticles are placed in a vacuum for approximately 10 minutes. A solution of 1,2-Distearoyl-sn-Glycero-3-Phosphoethanolamine-N-[Methoxy(Polyethylene glycol)-2000] (Mpeg 2000), 1,2-Distearoyl-sn-Glycero-3-Phosphoethanolamine-N-[Maleimide(Polyethylene Glycol)2000] (Mpeg 2000 Maleimide), and 1,2-Dioleoyl-sn-Glycero-3-Phosphoethanolamine-N-(Lissamine Rhodamine B Sulfonyl) is made with a 55:4:1 molar ratio respectively in chloroform (36.72 Ml per gram nanoparticles), where the amount of Mpeg 2000 phospholipids is given by the following formula:
M_{Mpeg2000}=1.019*Mₛₐₘₚₗₑ*R_{lipid}²/R³
where M_{Mpeg2000} is the mass of Mpeg 2000 lipids used in grams, Mₛₐₘₚₗₑ is the mass of the nanoparticle sample in grams, R is the radius of the nanoparticles in nm, and R_{lipid} is the phospholipid radius in nm (approximately R+2). The conjugation can be done with half the calculated value of phospholipids, but the coating process is more successful and that there is a significant reduction in the amount of precipitation when the higher amount is used. However, if the amount is increased even higher, there does not appear to be any additional improvement in the coating of the nanoparticles. For unconjugated nanoparticles, the coating can be done without the Mpeg 2000 Maleimide phospholipids, or with only the Mpeg 2000 or Mpeg 750 phospholipids. Additionally, it is believed that adding amine functionalized Mpeg 2000 phospholipids to the phospholipids coating will improve the ability of the coated nanoparticles to enter cells. The phospholipid solution is shaken and then added to the dried nanoparticles. The resulting nanoparticle solution is then vortexed for 1 hour. Once thoroughly mixed, the solution is transferred to a round bottom flask, where the chloroform is evaporated off. It is important to evenly coat the sides of the flask to prevent aggregation during hydration. Once all the chloroform has been evaporated, approximately 1 Ml of water is added per 0.01 g of nanoparticle. However, the amount of water added can be altered to obtain the desired concentration of nanoparticles. Additionally, the coated nanoparticles can be dispersed into a phosphate buffer solution using the same method but water appears to have slightly less precipitation. The solution is swirled in the flask for approximately 5 to 10 minutes until all of the nanoparticles have been dispersed in the water. The flask is then sonicated at room temperature for 30 minutes. If the nanoparticles are not being conjugated, they can be purified and transferred into a phosphate buffer solution by dialysing them overnight in the buffer solution, this will removed any excess phospholipids or precipitate.
Conjugating the Nanoparticles to Antibodies

For optimal conjugation, it is important to conjugate the nanoparticles to antibodies right after coating them with the phospholipids. The preparation method used on the antibodies is from the datasheet for Traut's Reagent (Pierce). To prepare the antibodies for conjugation, the antibodies are first reconstituted to a 10 mg/Ml concentration with a 3.5 Mm EDTA solution (water or PBS depending on the antibody instructions). 46 μL of 14 Mm Traut's Reagent in PBS is then added per Ml of antibody to modify the amine groups on the antibody into sulfhydral groups. The solution is allowed to incubate at room temperature for 1 hr. and then dialyzed for 1 hr. in a 3.5 Mm EDTA solution in PBS. After dialysis, the nanoparticles are mixed with the modified antibodies in a ratio of 7:1 nanoparticles to antibodies. The conjugation appears complete after about an hour, but the nanoparticle-antibody solution can be allowed to mixed overnight at 4° C. to ensure a complete reaction between the maleimide phospholipids on the nanoparticles and the sulfhydral groups on the antibodies. The conjugated nanoparticles are then dialyzed for 1 hr. in PBS.

What is claimed is:

1. A method for producing an iron oxide nanoparticle, the method comprising injecting iron pentacarbonyl into a reaction mixture, wherein the reaction mixture comprises oleic acid and trioctylamine (TOA), and wherein the reaction mixture is at a temperature of from about 180° C. to about 220° C.

2. The method of claim 1, wherein the reaction mixture is at a temperature of from about 190° C. to about 210° C.

3. The method of claim 1, wherein the reaction mixture is at a temperature of from about 195° C. to about 205° C.

4. The method of claim 1, wherein the reaction mixture is at a temperature of from about 198° C. to about 202° C.

5. The method of claim 1, wherein the reaction mixture is at a temperature of about 200° C.

6. The method of claim 1, wherein the reaction mixture consists of oleic acid and trioctylamine (TOA).

7. The method of claim 1, wherein the reaction mixture consists essentially of oleic acid and trioctylamine (TOA).

8. The method of claim 1, further comprising encapsulating the iron oxide nanoparticle in a phospholipid micelle, thereby making the iron oxide nanoparticle water-soluble.

9. The method of claim 1, wherein the nanoparticle is from about 2 to about 20 nanometers.

10. The method of claim 1, wherein the nanoparticle is about 5 nanometers.

11. The method of claim 1, wherein the nanoparticle comprises maghemite.

12. The method of claim 8, wherein the micelle comprises polyethylene glycol, methoxypolyethylene glycol 2000 (Mpeg 2000), Mpeg 2000 maleimide, 1,2-Diacyl-sn-Glycero-3-Phosphoethanolamine-N-[Methoxy(Polyethylene glycol)-350], 1,2-Diacyl-sn-Glycero-3-Phosphoethanolamine-N-[Methoxy(Polyethylene glycol)-750], 1,2-Diacyl-sn-Glycero-3-Phosphoethanolamine-N-[Methoxy(Polyethylene glycol)-2000], 1,2-Dipalmitoyl-sn-Glycero-3-Phosphocholine, cholesterol, or any combination thereof

13. The method of claim 8, wherein the micelle comprises from about 0.1% to about 10% functionalized phospholipids.

14. The method of claim 13, wherein the micelle comprises about 1% functionalized phospholipids.

15. The method of claim 14, wherein the functionalized phospholipids comprise thiol-functionalized phospholipids, amine functionalized phospholipids, or any combination thereof.

16. The method of claim 15, wherein the amine functionalized phospholipids comprise DSPE-PEG(2000)Carboxylic Acid, DSPE-PEG(2000)Maleimide, DSPE-PEG(2000)PDP, DSPE-PEG(2000)Amine, DSPE-PEG(2000)Biotin, or any combination thereof.

17. The method of claim 15, wherein the thiol-functionalized phospholipids comprise phophatidylthioethanol (PTE).

18. The method of claim 8, wherein the micelle comprises about 99% Mpeg750 and about 1% PTE phospholipids.

19. The method of claim 8, wherein the micelle further comprises phospholipids labeled with a fluorescent marker.

20. The method of claim 19, wherein the fluorescent marker comprises fluorescein.

21. The method of claim 19, wherein the phospholipids comprise 1,2-dioleoyl-sn-glycero-3-phosphoethanolamine-N-(Lissamine Rhodamine B Sulfonyl).

22. A method for conjugating a water-soluble iron oxide nanoparticle to a target molecule, the method comprising
(a) reacting a target molecule with a crosslinking agent, thereby forming a target molecule-crosslinking agent complex; and
(b) reacting a water-soluble iron oxide nanoparticle to the complex of step (a).

23. A method for conjugating a water-soluble iron oxide nanoparticle to a target molecule in the absence of a crosslinking agent, wherein the nanoparticle is conjugated directly to the target molecule.

24. The method of claim 22 or 23, wherein the target molecule comprises a therapeutic agent.

25. The method of claim 22 or 23, wherein the target molecule comprises a polypeptide, a nucleic acid, or a small molecule.

26. The method of claim 22 or 23, wherein the target molecule comprises an antibody.

27. The method of claim 26, wherein the antibody comprises an anti-insulin antibody.

28. The method of claim 20, further comprising concentrating the complex of step (a) before performing step (b).

29. The method of claim 22, wherein the crosslinking agent comprises a heterobifunctional crosslinking agent.

30. The method of claim 22, wherein the crosslinking agent comprises SMPT (4-succinimidyloxycarbonyl-α-methyl-α-(2-pyridylditio)toluene), sulfo-LC-SMPT (sulfosuccinimidyl-6-(α-methyl-α-(2-pyridylthio)toluamido) hexanoate, Traut's reagent (2-Iminothiolane•HCl), or any combination thereof.

31. A nanoparticle-target molecule conjugate prepared by the method of claim 22 or 23.

32. A method for detecting a cell of interest in a subject, the method comprising administering to the subject an effective amount of an iron oxide nanoparticle-antibody conjugate, wherein the antibody specifically binds to the cell.

33. A method for detecting a polypeptide in a subject, the method comprising administering to the subject an effective amount of an iron oxide nanoparticle-antibody conjugate, wherein the antibody specifically binds to the polypeptide.

34. The method of claim 32 or 33, wherein the nanoparticle is detected by magnetic resonance imaging or fluorescence imaging.
